(19) United States
(12) Patent Application Publication
(10) Pub. No.: US 2004/0081975 A1
(43) Pub. Date: Apr. 29, 2004

(54) Method for the characterization and/or identification of genomes

(76) Inventor: Jurg E. Frey, Wadenswil (CH)

Correspondence Address: Cooper & Dunham, 1185 Avenue of the Americas, New York, NY, 10036, US

(21) Appl. No.: 10/380,774

(22) PCT Filed: Sep. 12, 2001
(86) PCT No.: PCT/IB01/01651

(30) Foreign Application Priority Data
Sep. 18, 2000 (CH) 1806/00

(51) Int. Cl. C12Q 1/68

(52) U.S. Cl. 435/6

(57) ABSTRACT

The present invention relates to a method for the characterization and/or identification of genomes and target organisms, respectively, wherein the presence or absence of few or many nucleic acid sequences is determined in parallel in a sample of a biological organism and the resulting pattern is compared to patterns saved in an electronic databank by means of specific cluster algorithms and statistical methods.

### CROSS REFERENCES TO RELATED APPLICATIONS

This application claims the priority of Swiss patent application 1806/00, filed Sep. 18, 2000, the disclosure of which is incorporated herein by reference in its entirety.

### TECHNICAL FIELD

The present invention relates to a nucleic acid-based method for the characterization and identification of genomes. Said method enables the identification of nucleic acid containing organisms of all taxonomic levels.

### BACKGROUND ART

Besides the well established antibody based diagnostic methods nucleic acid based diagnostic methods have recently developed to a new standard both in medicine and in agricultural research.

The nowadays available methods of molecular and nucleic-acid based diagnosis are mainly based on the Polymerase chain reaction (PCR; Saiki et al., 1986). A reliable and reproducible identification with this method is possible if some information about short nucleic acid segments of each organism to be identified, the primer sequences, are known. Since said primer sequences are unknown for organisms that are genetically not analyzed or barely analyzed, an optimization of the method for each organism has normally to be carried out. Two methods are mainly used to characterize anonymous genomes, namely RAPD (random amplified polymorphic DNA) and AFLP (amplified fragment length polymorphism). The RAPD method can easily be carried out but there are problems concerning its reproducibility (Pérez et al., 1998). On the other hand AFLP shows a good reproducibility but is technically demanding if only small amounts of DNA are available (Mueller and LaReesa Wolfenbarger, 1999).

Further diagnostic methods are based on the microarray technology, where a large number of single analyses can be carried out in parallel on a two dimensional array (Brown and Botstein, 1999). These days said method is used to characterize single genotypes whereby for said use well defined DNA sequences of the genotype to be identified are used.

In agricultural diagnostics the use of the above described methods is hampered by a serious problem: the large number of bred animal species or animal races and of cultural plants worth protecting comprises a huge number of organisms to be identified. Among these organisms are organisms with unknown genomes (Frey and Frey, 1997) which can not be identified with the existing methods.

There is therefore an urgent need for a method, which allows an easy characterization and/or identification of genomes.

### DISCLOSURE OF THE INVENTION

Hence, it is a general object of the present invention to provide a method for the characterization and/or identification of genomes and target organisms, respectively, wherein the presence or absence of some or many nucleic acid sequences is detected in parallel in a probe of a biological organism and the resulting pattern is compared with patterns saved in an electronic databank by means of specific cluster algorithms and statistical methods.

Said method has several advantages compared to the currently used methods for the characterization of unknown genomes: No knowledge about the genome is necessary and small amounts of starting material (DNA or RNA) are sufficient. Furthermore, the method can easily be carried out and can be organized economically with respect to time and finance.

The present invention allows detecting the presence or absence of some or many different polynucleotide sequences and thereby permits the generation of a two dimensional pattern which is diagnostic i.e. a pattern that is characteristic for one or several organisms and which allows the explicit identification of said organism by comparison with patterns saved in a database.

A second exemplary application of the present invention is the characterization of genetic markers for phenotypically detectable features. The large number of anonymous primers with which a genome can be examined simultaneously permits a very efficient screening for molecular markers of interesting features. For example, markers for genes which confer resistance to pesticides can be found in pests. In plants markers for resistance genes against pests or quality features can be found.

### BRIEF DESCRIPTION OF THE DRAWINGS

The invention will be better understood and objects other than those set forth above will become apparent when consideration is given to the following detailed description thereof. Such description makes reference to the annexed drawings, wherein:

FIG. 1 shows an exemplary scheme of the method and

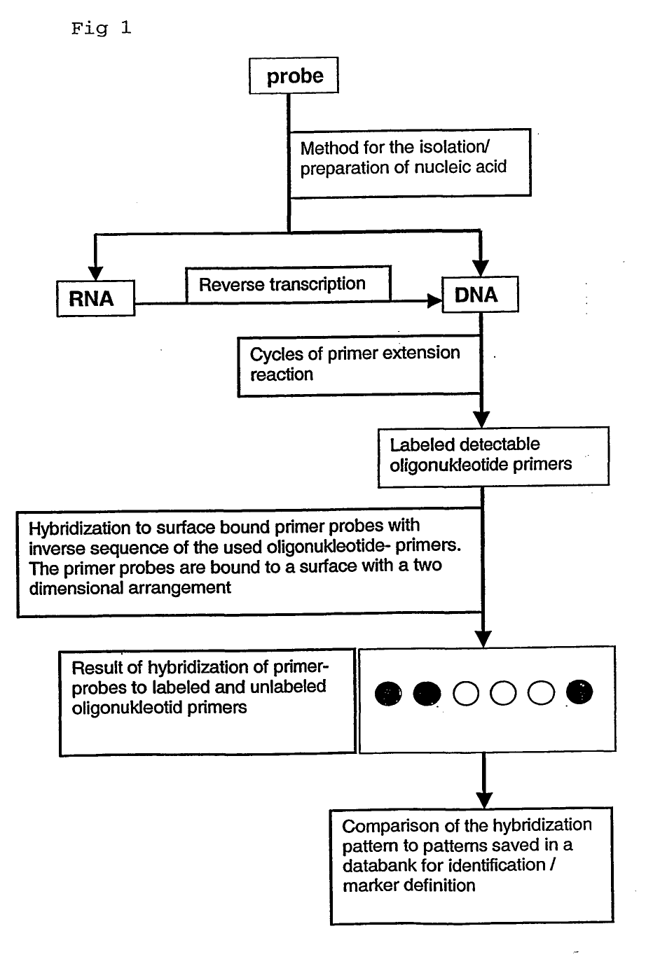

FIG. 2 shows a cluster diagram as a result of an assay with oligonucleotides of 12 nt length and 70% G/C content.

### MODES FOR CARRYING OUT THE INVENTION

The method of the present invention comprises the following steps:

A biological sample of an organism to be identified e.g. blood or a tissue sample, is processed to prepare the nucleic acid for the following chemical reaction. In case of a tissue sample this can be done using one of the established methods for mechanical disruption of the tissue followed by purification of the nucleic acid. If the isolated nucleic acid is a RNA, the RNA is in a first step transcribed to a DNA in a reaction with a reverse transcriptase.

In the next step at least one oligonucleotide primer, preferably up to a dozen, more preferably up to 1000, even more preferably up to 10'000 and most preferably more than 10'000 oligonucleotide primers are added together with part of the purified nucleic acid (now DNA) to a reaction mixture. The used oligonucleotide primers can comprise oligonucleotides with a random sequence and/or a sequence which is complementary to a target sequence of the DNA in the probe.

Preferably all oligonucleotide primers have within certain limits a uniform length, a uniform G/C content and a uniform melting temperature to allow extension of a large portion of the oligonucleotide primers under appropriate conditions. In addition to the compounds necessary for a oligonucleotide Primerextension reaction (mini sequencing reaction) the reaction mixture comprises one or several labeled didesoxynucleotide triphosphates (ddNTPs). If several different ddNTPs are used e.g. ddATP together with ddGTP, the single ddNTPs can be labeled with different markers. If all possible ddNTPs are labeled with fluorescence dyes, preferably each single ddNTP with a different fluorescence dye, the method can be used for the examination of SNPs (single nucleotide polymorphism). Alternatively, a mixture of ddNTPs and desoxynucleotide triphosphates (dNTPs) can be used whereby either the ddNTPs and/or the dNTPs are labeled. In the primer extension reaction DNTP and ddNTP analogs can be used as well. Suitable markers are e.g. chromophores, fluorophores and radioactive material. Preferably the ddNTPs or dNTPs are e.g. labeled with a fluorescence dye.

The resulting reaction mixture is adjusted to a temperature which allows that hybridization of the oligonucleotide primers to complementary DNA segments of the DNA to be analyzed can occur. Those oligonucleotide primers which find a complementary target sequence on the DNA hybridize to said target sequence. Said primers serve as primers in an extension reaction wherein the primers are extended by a heat stable polymerase which is as well present in the reaction mixture. In said extension reaction the oligonucleotide primer is extended by a labeled, preferably fluorescence labeled, didesoxynucleotide which is complementary to the nucleotide of the target sequence following the oligonucleotide primer sequence. When a mixture of ddNTPs and dNTPs is used the primer extension reaction is only interrupted after a ddNTP has been incorporated into the extended Primersequence.

The oligonucleotide primer which is extended by at least one labeled, preferably fluorescence labeled, nucleotide is dissociated from the target sequence by heating. A further round of primer extension is initiated by cooling down to hybridization temperature. At hybridization temperature a new set of oligonucleotide primers can anneal to the corresponding complementary sequences of the target DNA and the polymerase can add to each of the annealed primers a corresponding labeled, preferably fluorescence labeled, didesoxynucleotide and/or desoxynucleotide. Said cycle can be repeated several times and leads to a signal amplification for each primer with a corresponding complementary target sequence according to the rule (number of copies of target sequences times number of cycles). If for example there are 1000 copies of a complementary target sequence for a particular primer on the DNA in the sample then the extension reaction has generated about 50'000 color labeled copies of the primer after 50 cycles.

After completion of the labeling reaction it is determined for each primer present in the reaction whether there was a complementary target sequence on the probe DNA. If there was a complementary sequence present on the probe DNA an extension of the primer by at least one labeled, preferably fluorescence labeled, desoxynucleotide and/or didesoxynucleotide has occurred. For this purpose, for each primer used in the extension reaction an oligonucleotide having a sequence that corresponds to the complementary sequence of the primer, hereinafter called primer probe (PP), is required. Said PP is preferably at its 5′ end complementary to the oligonucleotide primer used and has at its 3′ end an extension allowing coupling to a substrate. Said 3′ end extension allowing coupling to a substrate is or comprises an anchorage. A suitable 3′ extension is e.g. a biotin molecule which allows a stable coupling to a substrate.

In a preferred embodiment of the present invention there is a nucleotide tail between the sequence complementary to the oligonucleotide primer and the anchorage e.g. a biotin molecule, in-order to allow a better hybridization of the PP with the corresponding oligonucleotide primer. Said substrate can e.g. be the surface of a microtiter plate well coated with a coupling allowing substance or a tube system in which said PPs are sequentially arranged. Such a system is e.g. the streptavidin—biotin bond. An oligonucleotide that is able to bind to a surface is e.g. prepared by modifying its 3′ end with a biotin molecule which can bind to a streptavidin molecule (affinity binding) and said streptavidin molecule is coupled to a surface. In this system the oligonucleotide is stably connected with the surface. The PP for each oligonucleotide primer used in the reaction can e.g. be coupled to the surface of a separate microtiter plate well, the surface of a microarray or can stably be coupled to another surface.

In a preferred embodiment of the present invention the number of surfaces corresponds to the number of primers used in the reaction wherein each of said surfaces carries a single PP specific for a primer. If for example 96 different primers were added to the reaction it is possible to analyze the reaction in a 96 well microtiter plate wherein each well contains a specific PP. For this purpose an aliquot of the reaction is e.g. added to each well. In a hybridization reaction the PP coupled to the surface of said well can then hybridize to its corresponding oligonucleotide primer. The advantage of the system with regard to the hybridization reaction is that the different oligonucleotide primers differ only in one nucleotide (if in the primer extension reaction only ddNTPs were present) or in a few nucleotides (if a mixture of ddNTPs and dNTPs were used). When in the primer extension reaction only ddNTPs were used, the labeled oligonucleotide primers are extended by a single labeled nucleotide. When a mixture of ddNTPs and dNTPs was used, the labeled oligonucleotide primers were extended by at least one, usually more than one nucleotide wherein one or several of the added nucleotides can be labeled. This procedure allows that uniform hybridization conditions can be used and therefore a very good reproducibility of the system can be achieved. After said hybridization reaction the substrate surfaces e.g. wells are washed to remove all oligonucleotide primers that did not hybridize.

In an another preferred embodiment of the present invention the primer probes are sequentially arranged in a closed tube system. The arrangement of the primer probes on a two dimensional microarray has the following disadvantage: the labeled or unlabeled primers of the hybridisation solution spread over the whole microarray surface and are in contact with all primer probes. Since the primers are homogenously distributed in the hybridisation solution only a small proportion of a labeled primer finds its corresponding PP. This results in a dilution effect weakening the signal of the labeled primers. The advantage of a tube system is that all primers get in close contact with their complementary PPs since said PPs are sequentially arranged and the whole hybridisation solution can be passed through the tube system. The flow of the hybridisation reaction can be unidirectional or bidirectional and the hybridisation reaction can be passed through the tube system once or more than once. The control of the temperature as well-as of the flow rate through the tube system allow an optimal control of the hybridisation whereby the reproducibility of the reaction is optimised. The spatial arrangement of the tube system is only determined by technical factors e.g. the used system for detection of the hybridisation and said tube system can be two dimensional or three dimensional.

After completion of hybridization the substrates bound to the PP are subjected to a detection test to determine which primers have been extended in the extension reaction. If the used ddNTPs and/or dNTPs were labeled with a fluorescence dye and a microtiter platewas used as substrate, then it is possible to determine whether an oligonucleotide primer that hybridized to a well contains a fluorescence labeled extension product by means of e.g. a fluorometer. When the different nucleotides used in the extension reaction were labeled with different fluorescence dies then it is possible to determine which of the four possible nucleotides was incorporated in a certain primer. Fluorescence can only be detected in wells where the PP have bound an oligonucleotide primer which has found a complementary region on the probe DNA and therefore said primer has incorporated in the extension reaction a fluorescence labeled didesoxynucleotide. When the wells are in a fixed arrangement to each other as for example in a microtiter plate, then the absence or presence of fluorescence in the wells generates a pattern. Said pattern is diagnostic for the probe DNA and can therefore be used for the identification.

A preferred embodiment of the tube system where the hybridisation reaction takes place, allows that the spatial arrangement of the hybridisation system can be chosen arbitrarily and said system nevertheless allows that a detection system without non-fixed parts focussing on a single detection area can be installed. In such an embodiment of the tube system the PPs represent small areas which are sequentially fixed to an elongated, thin fibre or lamella-like substrate (instead of fixing the PPs to a microarray surface). Said substrate is then incorporated into a tube system in which the hybridisation reaction takes place as described above. After completion of hybridisation the substrate can be removed from the tube system and can be subjected to a detection test in order to sequentially determine the status of each single PP area (labeled or unlabeled).

The characterization and/or identification of the probe DNA is the last step of the process of the present invention. If a microtiter plate and many oligonucleotide primers are used the identification of the probe DNA is preferably done by comparison of an analysis of the similarity of the generated pattern with known patterns from a databank. For this purpose various statistic programs containing cluster algorithm can be used.

The precision of the identification can e.g. be improved when in a selection process the patterns of randomly selected subsets of positive wells are compared to corresponding patterns in a databank. The advantage of said process is that even deviating patterns can be classified correctly. For example deviations from type patterns contained in a databank wherein said deviations are based on differences between different populations can be compensated. It is as well possible to recognize unknown taxa and the relationship of said unknown taxa to known groups can be roughly determined.

The present invention is now further illustrated by means of examples.

1. Verification of the Functional Principle in a Computer Simulation with 10'000 Primers

Requirements: the complete genome sequences of 22 microorganisms were downloaded from Genbank (see table 1). Based on literature dealing with genetic diversity of Escherichia coli (Whittam and Ake, 1993) the genome of said species was then mutated by the computer. The following parameters formed the bases of the process: According to Whittam and Ake (1993) the proportion of polymorphic nucleotides in E. coli based on a set of 11 genes averages 7.4%. This means that on an average one out of 14 nucleotides is polymorphic. The polymorphism for different genes can vary from 1.3 to 13.1% i.e. the difference factor is 10. Accordingly, three gene types were designated, PGL with a low grade DNA polymorphism of 2.5%, PGM with a medium grade DNA polymorphism of 7.0% and PGH with a high grade DNA polymorphism of 11.5%. A gene size of 1200 bp was assumed. This value corresponds to the rough average of genes examined by Whittman and Ake (1993). The percentage of each gene type was as well chosen according to the results of Whittman and Ake (1993). The parameters are summarized in table 2.

TABLE 1
List of examined species with acces-
sion number (genebank) and genome size (bp).

| | | | Genome |
|---|---|---|---|
| | abbre- | Genebank | size |
| Species | viation | Accession No | (bp) |
| Aquifex aeolicus | AQAEOL | AE000657 | 1551335 |
| Archaeoglobus fulgidus | ARFULG | AE000782 | 2178400 |
| Bacillus subtilis | BSU_ORI | AL009126 | 4214814 |
| Borrelia burgdorferi | BOBURG | AE000783 | 910724 |
| Chlamydia pneumoniae | CHPNEU | AE001363 | 1230230 |
| Chlamydia trachomatis | CHTRAC | AE001273 | 1042519 |
| Escherichia coli K-12 | ECO_ORI | NC_000913 | 4639221 |
| MG1655 | | | |
| Haemophilus influenzae | HAINFL | L42023 | 1830138 |
| Helicobacter pylori | HEPYLO | AE000511 | 1667867 |
| Methanobacterium thermo- | METHER | AE000666 | 1751377 |
| autotrophicum | | | |
| Methanococcus jannaschii | MEJANN | L77117 | 1664970 |
| Mycobacterium tuberculo- | MYTUBE | AL123456 | 4411529 |
| sis | | | |
| Mycoplasma genitalium | MYGENI | L43967 | 580074 |
| Mycoplasma pneumoniae | MYPNEU | U00089 | 816394 |
| Pyrococcus abyssi | PYABYS | AJ248283-7/ | 1500250 |
| | | AL096836 | |
| Pyrococcus horikoshii | PYHORI | Pyro_h | 1738505 |
| Rickettsia prowazekii | RIPROW | AJ235269 | 1111523 |
| Synechocystis PCC6803 | SYNESP | AB001339 | 3573470 |
| Thermotoga maritima | THMARI | AE000512 | 1860725 |
| Treponema pallidum | TRPALL | AE000520 | 1138011 |
| Saccharomyces cerevisiae | SC_TOT | NC_001133-48 | 12069247 |

TABLE 2
Parameters for the generation of
computer generated virtual bacterial strains. The average
gene size is 1200 base pairs (bp). The genome size of
both genomes had to be changed slightly (<0.02%) for com-
puter analysis. Grade of polymorphism of gene type PGL:
low, PGM: medium, PGH: high.

| | | | | Average per- |
|---|---|---|---|---|
| | | | | centage of |
| | | | | polymorphic |
| | | | Mutations per | Nukleotide |
| | N | Percent | Gene | sites |
| E. coli | | | | |
| Gene | 966 | 25,0 | 30 | 0,0250 |
| type PGL | | | | |
| Gene | 1611 | 41,7 | 84 | 0,0700 |
| type PGM | | | | |
| Gene | 1289 | 33,3 | 138 | 0,1150 |
| type PGH | | | | |
| Total | 3866 | 100 | 88.5 | 0.0738 |
| B. sub- | | | | |
| tilis | | | | |
| Gene | 874 | 24,9 | 30 | 0,0250 |
| type PGL | | | | |
| Gene | 1487 | 42,3 | 84 | 0,0700 |
| type PGM | | | | |
| Gene | 1152 | 32,8 | 138 | 0,1150 |
| type PGH | | | | |
| Total | 3513 | 100 | 88.3 | 0.0736 |

Computer programs: In the following process the used programs were either self made or commercially available software (Microsoft Excel, Microsoft Word)

1) Generation of all possible oligonucleotides of a defined length and a defined G/C content. The program generates all sequence combinations that are possible with the chosen parameters. With longer oligonucleotides several hundred million combinations are possible.

2) Generation of a list of 10'000 random numbers which as addresses of all oligonucleotides with a defined length and G/C content hit a random selection of 10'000 candidates.

3) Generation of the virtual strains of E. coli and Bacillus subtilis (B. subtilis) using the parameters of table 2. For this purpose a table was made which contains for each gene the assignment to a polymorphy group and random addresses indicating the nucleotides to be mutated. For each genome of both bacteria species six virtual strains were generated on the basis of said table. Three of the strains were generated with different random addresses of the nucleotides to be mutated and the other three generated strains are characterized in that all their genes have a low, medium or high grade of polymorphy, respectively.

4) Testing for presence/absence of each of the 10'000 oligonucleotid candidates in each of said strains of E. coli and B. subtilis as well as in all other microorganism genomes included in the analysis (table 2). The result is a matrix of 1 (present) or 0 (not present), respectively, for each oligonucleotide and all tested genomes.

5) Cluster analysis by means of the matrix for the detection of similarity between the different genomes generated under item 4.

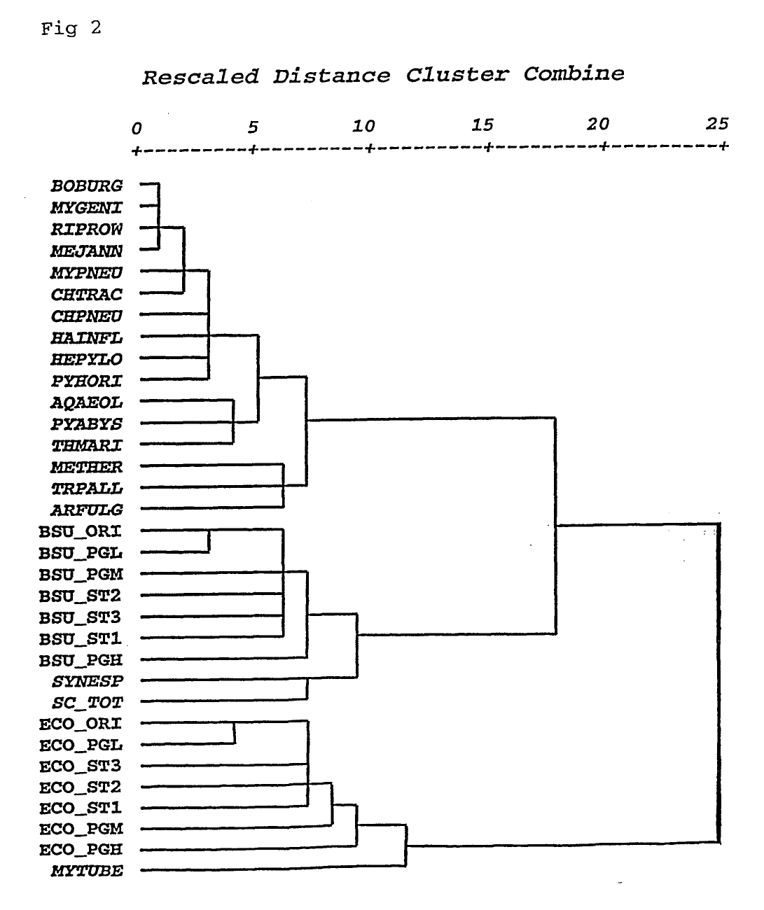

Result: If the correct parameters are chosen (e.g. length and/or G/C content of the oligonucleotide) then all generated virtual strains of E. coli should form with the original sequence a group which differs clearly from the other genomes. The same is true for the virtual strains of B. subtilis. FIG. 2 shows that this is fulfilled. This demonstrates the use of the principle. A similar high degree of assignment of strains to single species can as well be achieved with other sets of 10'000 randomly chosen oligonucleotides of 12 bp length and longer. This proves that the method is very reliable.

FIG. 2 shows a dendrogramm of the cluster analysis of the data matrix (presence/absence) for 10'000 randomly selected oligonucleotides of 12 bp length and a G/C content of 70%. All computer generated strains of E. coli and B. subtilis were each assigned to the correct group. The similarity between strains is clearly shown by the finding that for both species the least mutated strains are closest located to the original strain and the most mutated strains show the biggest deviation.

2. Proof of the Functional Principle with Probes in Microtiter Format

Requirements: All steps needed for a successful carrying out of the method are well established in the field and have proven to be reliable. Nowadays many commercial kits are available for the preparation of DNA. Said kits allow even the extraction of problematic templates (e.g. Dneasy Plant Mini Kit, Qiagen Ltd). Those oligonucleotides or oligonucleotide primers, respectively, for which a hybridization sequence on the probe DNA exists, are extended in a primer extension reaction also known as mini sequencing reaction (e.g. Plastinen et al., 1997). Said method is as well established and there are kits available therefor (e.g. Snapshot, PEbiosystems Ltd). After the primer extension reaction the labeled oligonucleotide primers have to be detected. For this purpose the reaction mixture is added to a two dimensional arrangement of primer probes. Each of the primer probes has an inverse sequence to one of the used oligonucleotide primers. The primer probes can for example be on a microarray or in a microtiter plate and can for example be stably bound to the surface by an affinity binding. A suitable system is e.g. the Biotin—Streptavidin bond. Each microarray spot or each microtiter plate well contains only a single primer probe. Said method is widely used in the field of micro chip technology and has proven to be reproducible (e.g. Hacia et al., 1998).

Carrying out: In the following section the technical feasibility of the principle of the method of the present invention is shown.

For this purpose a precisely known genome sequence has to be used. All natural organisms, even within closely related relationship groups, are different. Furthermore, mutations which change a defined sequence can always occur. Since the precise knowledge of the base sequence is a necessary prerequisite for the test, the precisely known sequence of the cloning vector pGEM-3Zf(+) was chosen (accession No. X65306; IG0050). The sequence has a length of 3199 bp and is characterized in great detail. Two primers with a corresponding hybridization sequence on the template DNA (match) and two primers without a corresponding hybridization sequence on the template DNA (mismatch) were chosen. (orientation 5′-3′; BIOT: biotinylated at the 3′ end): Match primer 1: cagcgggtgttg (Seq. Id. No. 1), match probe 1: caacacccgctg-BIOT (Seq. Id. No. 2); match primer 2: ggaagggcgatc (Seq. Id. No. 3); match probe 2: gatcgcccttcc-BIOT (Seq. Id. No. 4); mismatch primer 1: cgtgcacgttgc (Seq. Id. No. 5), mismatch probe 1: gcaacgtgcacg-BIOT (Seq. Id. No. 6); mismatch primer 2: gcgcctcatgac (Seq. Id. No. 7), mismatch probe 2: gtcatgaggcgc-BIOT (Seq. Id. No. 8. In a linear extension reaction (minisequencing) the primers are labeled by incorporation of a fluorescence labeled didesoxynucleotide which is complementary to the next nucleotide following the match primer sequence (using the Snapshot Kit of PEBiosystems). The mismatch primers do not find a complementary sequence on the template genome and are therefore not labeled. In the following Streptavidin coated microtiter plates are used. The biotinylated match or mismatch primer probes, respectively, are singly added to four wells e.g. probe 1 to well 1, probe 2 to well 2. After completion of the labeling reaction the reaction mixture is equally distributed to the four wells of the microtiter plate where the primerprobes of the match primers or the mismatch primers, respectively, are bound to the surface. In a hybridization reaction the bound primer probes of the match primers or the mismatch primers, respectively, bind the match primers or the mismatch primers, respectively, wherein said primers have the inverse sequence of the match primer probe or mismatch primerprobe, respectively. In well 1 the match primer probe 1 binds the match primer 1 and accordingly in the next three wells. In a control assay using a specific color medium which stains only double stranded DNA e.g. CybrGold(TM) the specificity of the hybridization is tested. All primer probe combinations are subjected to said control assay. The expected result is shown in table 3.

The unbound primers are then removed from the Streptavidin coated microtiter plate in a washing step. The sequence of the last step of the method, the detection of fluorescence in the reaction mixture, depends on the fluorescence detection system used. The microtiter plate can directly be analyzed in a fluorescence reader. Alternatively, the microtiter plate can be heated or can be treated with denaturing solutions in order to dissociate the hybridized and fluorescence labeled match primers from the match primer probes. The released fluorescence labeled match primers can then be collected and can be analyzed in a suitable fluorescence detection device e.g. by capillary electrophoresis in a ABI310 Genetic Analyzer (PE-Biosystems).

TABLE 3
Expected results of the analysis
with selected oligonukleotide primers and primer probes
using a fluorescence dye staining selectively double
stranded DNA; (+ = positives, − = negatives signal).

| | Match- | Match- | Mismatch- | Mismatch- |
|---|---|---|---|---|
| Probe | Probe 1 | Probe 2 | Probe 1 | Probe 2 |
| Match-primer 1 | + | − | − | − |
| Match-primer 2 | − | + | − | − |
| Mismatch-primer 1 | − | − | + | − |
| Mismatch-primer 2 | − | − | − | + |

Results: Color labeling of the used primers:

Two independent primer extension reactions with fluorescence labeled didesoxynucleotides were performed for each of the four used primers. For this purpose, the SnaPshot™ ddNTP primer Extension Kit of PEBiosystems was used according to the manufacturer's instructions and the reaction was then analyzed with a ABI310 Genetic Analyzer (PEBiosystems). Only those primers which found a corresponding sequence on the used template DNA (pGEM-3Zf(+)) were really labeled. The relative fluorescence values of two independent reactions each were for match primer 1 1327 and 639 units and for match primer 2 575 and 243 units. The corresponding values for the mismatch primers in both reactions were within the background noise (<20 units).

Hybridization of the Primers to the Immobilized Probes

The biotinylated probes were immobilized in Streptavidin coated microtiter plates (Black Combiplate 8 Streptavidin coated, Labsystems). 2 aliquots each of 20 μM biotinylated Probe was incubated in 50 μl binding and wash buffer (1M NaCl, 10 mM Tris-HCl pH 7.5, 1 mM EDTA) for 30 minutes with shaking (1000 rpm in Eppendorf Thermomixer Comfort) at room temperature and then washed four times with 50 μl of the same buffer. For hybridization 20 μM primer in 50 μl hybridization mixture (6×SSC: 0.9M NaCl, 0.9M Sodium citrate; 0.1% SCS, Denhard solution: 1% Ficoll, 1% Polyvinylpyrrolidon, 1% Bovine serum albumin) were added and incubated for 30 minutes at 40° C. and 1000 rpm. Then, the plate was washed four times with 601 hybridization wash buffer (0.1×SSC, 0.1% SDS). In or der to detect that the probes only bound its corresponding inverse primers 50 μl Cybr(R) Gold Nucleic Acid Gel Stain (Molecular Probes) was added and the relative fluorescence was measured in a fluorometer (Fluorskan Ascent FL, Labsystems). The measured values show that the immobilized probes preferably bind the matching primer (Table 4).

Table 4:Preferred hybridization of the primers with the inverse probes. The values show the average of the relative fluorescence measurement of two replications (each value is the average of 8 measured values; outliers with more than one standard deviation to the mean value were eliminated)

| | Match- | Match- | Mismatch- | Mismatch- |
|---|---|---|---|---|
| | Probe 1 | Probe 2 | Probe 1 | Probe 2 |
| Match-primer 1 | 1,27 | 0,49 | 1,23 | 0,99 |
| Match-primer 2 | 0,45 | 1,46 | 1,04 | 1,29 |
| Mismatch-primer 1 | 0,47 | 0,51 | 1,50 | 1,12 |
| Mismatch-primer 2 | 0,41 | 0,47 | 1,00 | 1,79 |

Detection of Hybridization of Labeled Primers

In order to demonstrate that the labeled primers hybridize, 15 μl extension reaction containing match primer 2 was added to one of the immobilized probes (match probe 2) and processed as described above. Afterwards the bound match primer 2 was dissociated from the probe by adding 20 μl denaturing solution (0.125M NaOH, 0.1M NaCl). 3 μl of said solution were analyzed in a ABI310. A fluorescence signal of 72 units was measured compared to a background signal of less than 4 units. This result shows that the labeled primers really-bind to the immobilized probes.

While there are shown and described presently preferred embodiments of the invention, it is to be distinctly understood that the invention is not limited thereto but may be otherwise variously embodied and practiced within the scope of the following claims.

### REFERENCES

Brown P O, Botstein D (1999) Exploring the new world of the genome with DNA microarrays. Nature Genetics Supplement 21: 33-37.

Frey J E, Frey B (1997) PCR-based diagnostics of agricultural pests and diseases. In: H.-W. Dehne et al. (eds.), Diagnosis and Identification of Plant Pathogens. Proceedings of the 4th International Symposium of the European Foundation for Plant Protection, pp. 23-28.

Hac [symbol] J G, Brody L C, Collins F S (1998) Applications of DNA chips for genomic analysis. Molecular Psychiatry 3: 4 [symbol] -492.

Lander E S (1999) Array of hope. Nature Genetics Supplement 21: 3-4

Mueller U G, LaReesa Wolfenbarger L (1999) AFLP genotyping and fingerprinting. Trends in Ecology and Evolution 14: 389-394.

Pastinen T, Kurg A, Metspalu A, Peltonen L, Syvanen A C (1997) Minisequencing: A specific tool for DNA analysis and diagnostics on oligonucleotide arrays. Genome Research 7: 606-614.

Pérez T, Albornoz J, Dominguez A (1998) An evalutation of RAPD fragment reproducibility and nature. Molecular Ecology 7: 1347-1357.

Saiki R K, Gelfand D H, Stoffel S, Scharf J, Horn G T, Mullis K B, Erlich H A (1988) Primer-directed enzymatic amplification of DNA with a thermostable Polymerase. Science 239: 487-491.

Whittam T S, Ake S E (1993) Genetic polymorphisms and recombination in natural populations of Escherichia coli. In: Mechanisms of molecular evolution, Naoyuki Takahata, Andrew G. Clark (eds.), Sinauer Associates, Tokyo, pp. 223-245.

1. Process for the characterization and/or identification of genomes comprising
hybridization of at least one oligonucleotide primer to a DNA sample of a genome to be characterized
extension of the annealed oligonucleotide primer in a minisequencing reaction in presence of at least one labeled didesoxynucleotide triphosphate and/or at least one labeled desoxynucleotide triphosphate
hybridization of the extension reaction to a primer probe wherein the sequence of said primer probe corresponds to the complementary sequence of said primer
detection of a bound extension product
characterization and/or identification of the genome by means of cluster algorithm programs.

2. Process according to claim 1 wherein more than one oligonucleotide primer, preferably up to a dozen, more preferably up to one thousand, even more preferably up to 10'000 and most preferably more than 10'000 primers are used.

3. Process according to claim 1 or 2 wherein the primers have a random nucleotide sequence.

4. Process according to claim 1 or 2 wherein the primers have a sequence that is complementary to a target sequence of the genome to be characterized.

5. Process according to any one of the preceding claims wherein a mixture comprising primers with random sequence and primers with a complementary sequence to a target sequence of the genome to be characterized is used.

6. Process according to any one of the preceding claims wherein the at least one primer has a defined length and/or a defined G/C content.

7. Process according to claim 1 or 2 wherein the at least one primer has a defined melting temperature.

8. Process according to any one of the preceding claims wherein the at least one didesoxynucleotide triphosphate and/or the at least one desoxynucleotide triphosphate is fluorescence labeled.

9. Process according to any one of the preceding claims wherein all 4 ddNTPs are fluorescence labeled, preferably each ddNTP with a different fluorophore.

10. Process according to any one of the preceding claims wherein the 5′ end of the primer probe corresponds to the complementary sequence of the used oligonucleotid primer and its 3′ end has an extension allowing the coupling to a substrate.

11. Process according to claim 10 wherein said 3′ end extension comprises or is an anchorage.

12. Process according to claim 11 wherein said anchorage is a Biotin molecule.

13. Process according to any one of claims 10 to 12 wherein the primer probe has between its 5′ end that corresponds to the complementary sequence of the used oligonucleotid primer and its 3′ end extension a nucleotide tail.

14. Process according to any one of claims 10 to 13 wherein the substrate is a surface of a microtiter plate well, a surface of a microarray or a fibre/lamella-like elongated substrate.

15. Process according to any one of claims 1 to 13 wherein the hybridisation reaction takes place in a closed tube system comprising the sequentially arranged primer probes fixed to an elongated substrate.

16. Process according to any one of the preceding claims wherein the probe DNA of the genome to be characterized is synthesized by a reverse transcriptase from RNA.
